(19) United States
(12) Patent Application Publication
(10) Pub. No.: US 2022/0283163 A1
(43) Pub. Date: Sep. 8, 2022

(54) Biomarkers for Detecting Secondary Liver Cancer

(71) Applicant: Erasmus University Medical Center Rotterdam, Rotterdam (NL)

(72) Inventors: Johannes Nicolaas Maria Ijzermans, Rotterdam (NL); Nick Arnold Van Huizen, Rotterdam (NL); Theo Marten Luider, Rotterdam (NL)

(21) Appl. No.: 17/636,516

(22) PCT Filed: Aug. 20, 2020
(86) PCT No.: PCT/NL2020/050519

§ 371 Date: Feb. 18, 2022

(30) Foreign Application Priority Data
Aug. 20, 2019 (NL) 2023672

(51) Int. Cl. G01N 33/574; G01N 33/68

(52) U.S. Cl. CPC G01N 33/57419; G01N 33/57438; G01N 33/6842; G01N 2800/7028

(57) ABSTRACT

The invention relates to a method for typing a subject for the presence or absence of a secondary liver cancer, comprising the steps of—measuring in a sample comprising peptides from a subject a peptide level for (i) a peptide comprising the amino acid sequence of SEQ ID NO:4 or a peptide comprising an amino acid sequence that has at least 90% sequence identity to the amino acid sequence of SEQ ID NO:4; and/or (ii) a peptide comprising the amino acid sequence of SEQ ID NO:1 or a peptide comprising an amino acid sequence that has at least 90% sequence identity to the amino acid sequence of SEQ ID NO:1; and—typing said subject for the presence or absence of said secondary liver cancer on the basis of the measured peptide level.

### FIELD OF THE INVENTION

This invention relates to the field of diagnostics, more specifically to the field of peptide biomarkers for detecting secondary liver cancer. More specifically, the invention relates to hydroxylated collagen natural occurring peptides (NOP) which allow for detection of secondary liver cancer in a subject. The invention also relates to uses of such biomarkers. The invention also relates to therapy, more specifically to the treatment of subjects suffering from secondary liver cancer that are typed according to a method as described herein.

### STATE OF THE ART

Colorectal cancer is the third most often diagnosed cancer in the Netherlands. Between 2010 and 2017, annually 10,000-16,000 new patients were diagnosed and 5000 patients died (Integraal Kankercentrum Nederland, 2018). In the Western world, the probability that a patient will develop metastases to the liver (colorectal liver metastases; CRLM) after curative surgery of the primary tumor is 20-40% (Riihimaki et al., Sci Rep. 6:29765 (2016); Figueredo et al., BMC Cancer; 3:26 (2003); Al-Asfoor et al., Cochrane Database Syst Rev. CD006039 (2018); Gregoire et al., Eur J Surg Oncol. 36:568-74 (2010); Grossmann et al., Colorectal Dis. 9:787-92 (2007)).

After curative surgery, a patient is offered an intensive 5-year follow-up program, which consists of regular computed tomography (CT) scans, ultrasound studies, and carcinoembryonic antigen (CEA) serum measurements to screen for CRLM (Grossmann et al., Colorectal Dis. 9:787-92 (2007); Locker et al., J Clin Oncol., 24:5313-27 (2006); Pita-Fernandez et al., Ann Oncol., 26:644-56 (2015)).

It was previously reported that a combination of serum CEA and a specific collagen natural occurring peptide (NOP) in urine can be used in detecting CRLM (sensitivity 85%, specificity 84%) (Lalmahomed et al., Am J Cancer Res., 6:321-30 (2016)). Even though the sensitivity and specificity of this combination is higher than the sensitivity and specificity of the currently used techniques, they still can be improved, which is beneficial when considering application in a clinical setting.

There is a need in the art for further biomarkers that can be used to detect secondary liver cancer such as CRLM.

It is an aim of the present invention to provide for such biomarkers, especially biomarkers that are hydroxylated collagen natural occurring peptides. Alternatively, it is an aim of the invention to improve on CEA serum measurements in secondary liver cancer detection. The availability of a robust test could strongly reduce the number of procedures in the follow-up period after surgical resection of the primary tumor in cancer patients.

### SUMMARY OF THE INVENTION

Therefore, the present invention provides in one aspect a method for typing a subject for the presence or absence of a secondary liver cancer, comprising the steps of—measuring in a sample comprising peptides from a subject a peptide level for (i) a peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4; and—typing said subject for the presence or absence of said secondary liver cancer on the basis of the measured peptide level. Alternatively, the present invention provides in one aspect a method for typing a subject for the presence or absence of a secondary liver cancer, comprising the steps of—measuring in a sample comprising peptides from a subject a peptide level for (i) a peptide comprising the amino acid sequence of SEQ ID NO:1 or a peptide comprising an amino acid sequence that has at least 90% sequence identity to the amino acid sequence of SEQ ID NO:1, and/or (ii) a peptide comprising the amino acid sequence of SEQ ID NO:4 or a peptide comprising an amino acid sequence that has at least 90% sequence identity to the amino acid sequence of SEQ ID NO:4; and—typing said subject for the presence or absence of said secondary liver cancer on the basis of the measured peptide levels.

The inventors unexpectedly identified three new hydroxylated collagen natural occurring peptides (NOPs) (hydroxylated collagen NOP peptide “GND” (SEQ ID NO:1), hydroxylated collagen NOP peptide “GPP” (SEQ ID NO:2) and hydroxylated collagen NOP peptide GER (SEQ ID NO:4)) that can be advantageously used in the detection of secondary liver cancer in a subject suffering from, or a subject having suffered from, cancer such as colorectal cancer (Table 3 and Table 5).

In addition, the inventors established that combined use of the peptide of SEQ ID NO:1 and carcinoembryonic antigen (CEA) allows for superior detection of secondary liver cancer in subjects suffering from or having suffered from cancer (Table 4 and FIG. 3). This combination proved to have a significantly higher predictive power than the previous model that was based on hydroxylated collagen NOP peptide “AGP” (SEQ ID NO:3) and CEA. The sensitivity increased from 80% to 92%, whereas the specificity increased from 80% to 90% in the validation set, which was an independently collected sample set. The sensitivity achieved with this new combination is at least 15-20% higher than that of the currently used techniques, ranging from 57% to 70%. The specificity is comparable to these techniques, ranging from 90% to 96%. Overall, the performance of this new combination is better than that of the currently used techniques. This property is clinically beneficial, because earlier detection of secondary liver cancers such as CRLM can be foreseen and reduces health care costs. A similar beneficial effect was observed when using the NOP peptide GER in combination with CEA (Example 2, Table 5).

In a preferred embodiment of said method for typing, the method for typing a subject further comprising the steps of—comparing said measured peptide level to a reference peptide level for (i) said peptide comprising the amino acid sequence of SEQ ID NO:1, SEQ ID NO:2 or SEQ ID NO:4 or (ii) said peptide comprising an amino acid sequence that has at least 90% sequence identity to the amino acid sequence of SEQ ID NO:1, SEQ ID NO:2 or SEQ ID NO:4; and—typing said subject for the presence or absence of a secondary liver cancer on the basis of the comparison of the measured peptide level and the reference peptide level.

In a preferred embodiment of said method of typing, the method of typing further comprises the steps of—comparing said measured peptide level to a reference peptide level for (i) said peptide comprising the amino acid sequence of SEQ ID NO:1 or said peptide comprising an amino acid sequence that has at least 90% sequence identity to the amino acid sequence of SEQ ID NO:1; and/or (ii) said peptide comprising the amino acid sequence of SEQ ID NO:4 or a peptide comprising an amino acid sequence that has at least 90% sequence identity to the amino acid sequence of SEQ ID NO:4; and—typing said subject for the presence or absence of a secondary liver cancer on the basis of the comparison of the measured peptide level and the reference peptide level.

In another preferred embodiment of said method for typing, wherein a peptide comprising SEQ ID NO:1 and a peptide comprising SEQ ID NO:4 are employed (in combination) in a method for typing of the invention, the method for typing further comprises the steps of—comparing said measured peptide levels to a reference peptide level for (i) said peptide comprising the amino acid sequence of SEQ ID NO:1 or said peptide comprising an amino acid sequence that has at least 90% sequence identity to the amino acid sequence of SEQ ID NO:1, and (ii) said peptide comprising the amino acid sequence of SEQ ID NO:4 or said peptide comprising an amino acid sequence that has at least 90% sequence identity to the amino acid sequence of SEQ ID NO:4; and—typing said subject for the presence or absence of a secondary liver cancer on the basis of the comparison of the measured peptide levels and the reference peptide level(s).

In another preferred embodiment of said method for typing, the subject is a subject suffering from, or a subject having suffered from, a primary cancer, preferably a primary colorectal cancer.

In a further preferred embodiment of said method for typing, said subject is a subject that suffered from a primary cancer, preferably a primary colorectal cancer, and in which the primary cancer was surgically resected.

In another preferred embodiment of said method for typing, said sample comprising peptides from a subject is a bodily fluid sample, preferably a urine sample, from said subject.

In a further preferred embodiment of said method for typing, said sample comprising peptides is a sample comprising collagen natural occurring peptides (NOPs).

In another preferred embodiment of said method for typing, said reference peptide level(s) is (are) measured in a sample comprising peptides from a reference subject not suffering from, or a reference subject not having suffered from, cancer.

In a further preferred embodiment of said method for typing, said subject, or said sample, is typed as having a secondary liver cancer when said peptide level is increased as compared to said reference peptide level.

In another preferred embodiment of said method for typing, said method further comprises the step of—measuring in a sample comprising proteins from said subject a carcinoembryonic antigen (CEA) protein level;—typing said subject for the presence or absence of a secondary liver cancer on the basis of the measured peptide level and the measured CEA protein level.

In a further preferred embodiment of said method for typing comprising measuring a CEA protein level, said proteins from said subject are proteins from a blood sample of said subject.

In a further preferred embodiment of said method for typing comprising measuring a CEA protein level, said subject, or said sample, is typed for the presence or absence of a secondary liver cancer by using the formula as indicated herein below:

$$Probability of having secondary liver cancer = \frac{1}{1 + {e}^{- 1 * ( - 24.1476 + 3.0365 * GND + 3.4647 * CEA )}}$$

wherein “GND” is the measured peptide level of the peptide of SEQ ID NO:1, or of a peptide comprising an amino acid sequence that has at least 90% sequence identity to the amino acid sequence of SEQ ID NO:1, and wherein “GND” is expressed in area under the peak (curve) as measured by mass spectrometry; and “CEA” is the measured CEA protein level expressed in ng CEA/ml serum.

In a further preferred embodiment of said method for typing comprising measuring a CEA protein level, said subject, or said sample, is typed for the presence or absence of a secondary liver cancer by using the formula as indicated herein below:

$$Probability of having secondary liver cancer = \frac{1}{1 + {e}^{- 1 * ( - 20.62 + 3.05 * CEA + 2.49 * GER )}}$$

wherein “GER” is the measured peptide level of the peptide of SEQ ID NO:4, or of a peptide comprising an amino acid sequence that has at least 90% sequence identity to the amino acid sequence of SEQ ID NO:4, and wherein “GER” is expressed in area under the peak (curve) as measured by mass spectrometry; and “CEA” is the measured CEA protein level expressed in ng CEA/ml serum.

In a further preferred embodiment of said method for typing comprising measuring a CEA protein level, said method further comprises the steps of—comparing said measured protein level to a reference CEA protein level; and—typing said subject for the presence or absence of a secondary liver cancer on the basis of the (i) comparison of the measured peptide level(s) and the reference peptide level(s) and (ii) comparison of the measured CEA protein level and the reference CEA protein level.

In a further preferred embodiment of said method for typing comprising measuring a CEA protein level, said reference CEA protein level is measured in a sample comprising proteins from a reference subject not suffering from, or a reference subject not having suffered from, cancer.

In a further preferred embodiment of said method for typing comprising measuring a CEA protein level, said subject, or said sample, is typed as having a secondary liver cancer when (i) said peptide level(s) is increased as compared to said reference peptide level(s) and (ii) said CEA protein level is increased as compared to said reference CEA protein level.

In another aspect, the invention provides a use of a (i) peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4, in typing a subject for the presence or absence of a secondary liver cancer. The invention also provides a use of (i) a peptide comprising the amino acid sequence of SEQ ID NO:1 or a peptide comprising an amino acid sequence that has at least 90% sequence identity to the amino acid sequence of SEQ ID NO:1 or (ii) a peptide comprising the amino acid sequence of SEQ ID NO:4 or a peptide comprising an amino acid sequence that has at least 90% sequence identity to the amino acid sequence of SEQ ID NO:4, in typing a subject for the presence or absence of a secondary liver cancer.

In an embodiment, said use involves a combination of a (i) peptide comprising the amino acid sequence of SEQ ID NO:1, SEQ ID NO:2 or SEQ ID NO:4 or (ii) peptide comprising an amino acid sequence that has at least 90% sequence identity to the amino acid sequence of SEQ ID NO:1, SEQ ID NO:2 or SEQ ID NO:4, and carcinoembryonic antigen (CEA) in typing a subject for the presence or absence of a secondary liver cancer. In an embodiment of such a use, a combination of a peptide comprising the amino acid sequence of SEQ ID NO:1, or a peptide comprising an amino acid sequence that has at least 90% sequence identity to the amino acid sequence of SEQ ID NO:1, and a peptide comprising the amino acid sequence of SEQ ID NO:4, or a peptide comprising an amino acid sequence that has at least 90% sequence identity to the amino acid sequence of SEQ ID NO:4, is employed in combination with carcinoembryonic antigen (CEA) in typing a subject for the presence or absence of a secondary liver cancer.

In a preferred embodiment of said method for typing, or said use, said secondary liver cancer is colorectal liver metastases (CRLM).

In another aspect, the invention provides a peptide comprising the amino acid sequence of SEQ ID NO:1, SEQ ID NO:2 or SEQ ID NO:4, or a peptide comprising an amino acid sequence that has at least 90% sequence identity to the amino acid sequence of SEQ ID NO:1, SEQ ID NO:2 or SEQ ID NO:4. A peptide comprising or consisting of the amino acid sequence of SEQ ID NO:1 or SEQ ID NO:4, or a peptide comprising or consisting of an amino acid sequence that has at least 90% sequence identity to the amino acid sequence of SEQ ID NO:1 or SEQ ID NO:4, are especially preferred, also in the methods as described herein. Therefore, the invention also provides a peptide comprising or consisting of the amino acid sequence of SEQ ID NO:1 or a peptide comprising or consisting of an amino acid sequence that has at least 90% sequence identity to the amino acid sequence of SEQ ID NO:1, or a peptide comprising or consisting of the amino acid sequence of SEQ ID NO:4 or a peptide comprising or consisting of an amino acid sequence that has at least 90% sequence identity to the amino acid sequence of SEQ ID NO:4.

In a preferred embodiment of said peptide, said peptide comprises or consists of the amino acid sequence of SEQ ID NO:1 or SEQ ID NO:4.

In another aspect, the invention provides a standard-of-care therapeutic agent against a secondary liver cancer for use in treating a subject typed as having a secondary liver cancer, preferably CRLM, according to a method for typing of the invention.

In another aspect, the invention provides a use of a standard-of-care therapeutic agent against a secondary liver cancer for the manufacture of medicament for treating a subject suffering from a secondary liver cancer; wherein said subject is typed as having a secondary liver cancer according to a method for typing according to the invention.

In another aspect, the invention provides a method for treating a subject suffering from a secondary liver cancer, comprising the steps of—performing a method for typing according to the invention;—administering a therapeutically effective amount of a standard-of-care therapeutic agent against a secondary liver cancer when said subject is typed as having a secondary liver cancer.

In another aspect, the present invention provides a method for measuring a peptide level, comprising the step of:—optionally, providing a sample comprising peptides from a subject;—measuring in a sample comprising peptides from a subject a peptide level for a peptide comprising the amino acid sequence of SEQ ID NO:1, SEQ ID NO:2 and/or SEQ ID NO:4, or a peptide comprising an amino acid sequence that has at least 90% sequence identity to the amino acid sequence of SEQ ID NO:1, SEQ ID NO:2 or SEQ ID NO:4. In an embodiment of such a method, a peptide level can be measured for a combination of (i) a peptide comprising the amino acid sequence of SEQ ID NO:1, or a peptide comprising an amino acid sequence that has at least 90% sequence identity to the amino acid sequence of SEQ ID NO:1, and (ii) a peptide comprising the amino acid sequence of SEQ ID NO:2, or a peptide comprising an amino acid sequence that has at least 90% sequence identity to the amino acid sequence of SEQ ID NO:2. Alternatively, a peptide level can be measured for a combination of (i) a peptide comprising the amino acid sequence of SEQ ID NO:1, or a peptide comprising an amino acid sequence that has at least 90% sequence identity to the amino acid sequence of SEQ ID NO:1, and (ii) a peptide comprising the amino acid sequence of SEQ ID NO:4, or a peptide comprising an amino acid sequence that has at least 90% sequence identity to the amino acid sequence of SEQ ID NO:4.

The invention also provides a method for measuring a peptide level, comprising the step of:—optionally, providing a sample comprising peptides from a subject;—measuring in a sample comprising peptides from a subject a peptide level for (i) a peptide comprising the amino acid sequence of SEQ ID NO:1 or a peptide comprising an amino acid sequence that has at least 90% sequence identity to the amino acid sequence of SEQ ID NO: 1 and/or (ii) a peptide comprising the amino acid sequence of SEQ ID NO:4 or a peptide comprising an amino acid sequence that has at least 90% sequence identity to the amino acid sequence of SEQ ID NO:4.

In the above-described methods for measuring a peptide level, the method may further comprise a step of—optionally, providing a sample comprising proteins from said subject;—measuring in a sample comprising proteins from said subject a carcinoembryonic antigen (CEA) protein level. In a preferred embodiment of said method for measuring a peptide level, said sample comprising peptides is a urine sample and said sample comprising proteins is a blood sample, preferably a serum or plasma sample.

### DETAILED DESCRIPTION OF THE INVENTION

The term “typing”, as used herein, refers to differentiating between, or stratification of, subjects on the basis of whether a secondary liver cancer such as CRLM is present or absent. The term also includes reference to diagnosis or detection of secondary liver cancers such as CRLM. Preferably, in a method for typing of the invention, the typing is based on a comparison of (i) the measured peptide level and (ii) a reference peptide level for said peptide comprising the amino acid sequence of SEQ ID NO:1, SEQ ID NO:2 or SEQ ID NO:4, or for said peptide comprising an amino acid sequence that has at least 90% sequence identity to the amino acid sequence of SEQ ID NO:1. SEQ ID NO:2 or SEQ ID NO:4.

The term “subject”, as used herein, refers to a mammal, more preferably a primate, most preferably a human. The term includes reference to a patient suffering, or having suffered from a malignant tumor, preferably wherein the malignant tumor is a primary (malignant) tumor, for instance selected from the group formed by breast cancer; colorectal cancer; kidney cancer; esophageal cancer; lung cancer; skin cancer; ovarium cancer; uterine cancer, including endometrial cancer and uterine sarcoma; brain cancer; pancreatic cancer and stomach cancer. It is well-known that primary (malignant) tumors of these cancer types can spread to the liver.

Preferably, in a method for typing of the invention, the subject is a patient suffering, or having suffered, from a primary (malignant) tumor. More preferably, the subject is a patient in which the primary (malignant) tumor was surgically resected. Even more preferably, the subject is a patient that received (or underwent) curative surgical resection of the primary (malignant) tumor. Preferably, the subject did not yet develop a secondary cancer, such as metastases, after surgical resection of the primary (malignant) tumor. Alternatively, the subject as described herein is a patient suffering, or having suffered, from a primary (malignant) tumor and is at risk of developing secondary live cancer. In principle, all cancer patients that received surgical resection of the primary (malignant) tumor are at risk of developing secondary liver cancer.

Preferably, in a method for typing of the invention, the subject is a subject suffering, or having suffered, from a primary (malignant) tumor that is a colorectal cancer. More preferably, the subject is a patient in which the primary (malignant) tumor, that is a colorectal cancer, was surgically resected. Even more preferably, the subject is a patient that received (or underwent) curative surgical resection of the primary (malignant) tumor that is a colorectal cancer. Preferably, the subject did not yet develop a secondary cancer, including metastases, after surgical resection of the primary (malignant) tumor that is a colorectal cancer. Alternatively, the subject as described herein is a patient suffering, or having suffered, from a primary (malignant) tumor that is a colorectal cancer and is at risk of developing secondary live cancer. In principle, all cancer patients that received surgical resection of the primary (malignant) tumor, that is a colorectal cancer, are at risk of developing secondary liver cancer.

The term “secondary liver cancer”, as used herein, includes reference to a cancer that is present in the liver, but that originated elsewhere in the body. For example, cancer may originate as colorectal cancer (primary malignant tumor), and the colorectal cancer cells may spread or metastasize to the liver to form liver cancer of colorectal origin (secondary liver cancer). Secondary liver cancer may originate from cancers including, but not limited to, breast cancer; lung cancer; colorectal cancer; brain cancer; kidney cancer; esophageal cancer; skin cancer; ovarium cancer; uterine cancer, including endometrial cancer and uterine sarcoma; pancreatic cancer and stomach cancer. Most preferably, the secondary liver cancer is colorectal liver metastases (CRLM).

The terms “primary tumor” and “primary cancer” are used interchangeably herein.

The term “colorectal liver metastases” or “CRLM”, as used herein, refers to a well-established clinical indication wherein metastases form in the liver of colorectal cancer patients. The liver is the most frequent site of metastasis in colorectal cancer patients.

The term “sample”, as used herein, refers to a sample that comprises peptides and/or proteins from a subject. The sample is preferably a bodily fluid sample. Such samples include, but are not limited to, sputum, blood, serum, plasma, urine, peritoneal fluid and pleural fluid. Most preferably, the sample is a urine sample when a peptide level of a peptide as described herein is to be measured, and is a serum sample when a CEA protein level is to be measured. Obtaining such samples is well within common general knowledge of the skilled person.

Preferably, the sample is a processed or prepared sample, such as a urine sample that is processed in order to be used in, or prepared for, a peptide or protein level measurement step. Such processing or preparing is routine and can for instance include a step wherein (collagen) natural occurring peptides (NOPs) are separated from other components of the sample, including small molecules, salts and proteins. This can for instance be done by using a protein recovery column such as a mRP C-18 Hi-Recovery Protein Column (4.6×50 mm) (Agilent, Amstelveen, the Netherlands) in combination with liquid chromatography. Subsequently, the peptide fraction, preferably NOP fraction, can be collected, dried, reconstituted (for instance with an aqueous liquid such as water, including 0.1% trifluoroacetic acid (TFA) in water), and/or analyzed using peptide or protein level measurement techniques, including mass spectrometry.

The terms “protein” and “peptide”, as used herein, refer to a polymer of amino acid residues (an amino acid sequence). These terms also includes reference to modified peptides or proteins, such as a stable isotope labelled (SIL) peptide. Preferably, when reference is made to a peptide herein, it refers to a hydroxylated collagen NOP peptide of the invention as described herein. Preferably, when reference is made to a protein, it refers to CEA.

The term “natural occurring peptide” or “NOP”, as used herein, includes reference to a peptide that naturally occurs in a subject. Preferably, such a NOP is a collagen NOP, i.e. a collagen-derived NOP, more preferably a hydroxylated collagen NOP, even more preferably a hydroxylated collagen NOP comprising the amino acid sequence according to any one of SEQ ID NO:1, SEQ ID NO:2 or SEQ ID NO:4, and most preferably a hydroxylated collagen NOP comprising the amino acid sequence of SEQ ID NO:1 or SEQ ID NO:4. An advantage of the NOP GER peptide of SEQ ID NO:4 is that it does not contain hydroxylated lysine residues. It is expensive to obtain SIL NOP peptides for reference purposes that have hydroxylated lysine residues. The term “NOP” includes reference to a hydroxylated NOP such as a hydroxylated collagen NOP.

The peptides used in a method for typing according to the invention are hydroxylated.

Hydroxylation is a process that introduces a hydroxyl group into an amino acid and is facilitated by enzymes called hydroxylases. The principal residue to be hydroxylated in peptides is proline, but other amino acid residues such as lysines can be hydroxylated as well. The hydroxylation occurs mostly at the γ-C atom, forming hydroxyproline (Hyp). In some cases, proline may be hydroxylated instead on its β-C atom. Lysine may also be hydroxylated on its δ-C atom, forming hydroxylysine (Hyl). These reactions may be catalyzed by multi-subunit enzymes prolyl 4-hydroxylase, prolyl 3-hydroxylase and lysyl 5-hydroxylase, respectively. Also cysteine, phenylalanine, tyrosine are examples of amino acids that may be hydroxylated.

In a method for typing according to the invention, a peptide level is determined for (i) a peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4. Preferably, the sequence identity is at least 91%, 92%, 93%, 94%, 95%, 97%, 98% or at least 99%. Preferably, the peptide comprising an amino acid sequence that has at least 90% sequence identity to the amino acid sequence of SEQ ID NO:1 has at least 1, 2, 3, 4, 5 or 6 hydroxylated amino acid residues in the manner as defined in SEQ ID NO:1. More preferably, the peptide comprising an amino acid sequence that has at least 90% sequence identity to the amino acid sequence of SEQ ID NO:1 has all 6 hydroxylated amino acid residues as defined in SEQ ID NO:1, and therefore the same hydroxylation pattern as defined in SEQ ID NO:1. Preferably, the peptide comprising an amino acid sequence that has at least 90% sequence identity to the amino acid sequence of SEQ ID NO:2 has at least 1, 2, 3 or 4 hydroxylated amino acid residues in the manner as defined in SEQ ID NO:2. More preferably, the peptide comprising an amino acid sequence that has at least 90% sequence identity to the amino acid sequence of SEQ ID NO:2 has all 4 hydroxylated amino acid residues as defined in SEQ ID NO:2, and therefore has the same hydroxylation pattern as defined in SEQ ID NO:2. Preferably, the peptide comprising an amino acid sequence that has at least 90% sequence identity to the amino acid sequence of SEQ ID NO:4 has at least 1, 2, 3, 4, 5, 6 or 7 hydroxylated amino acid residues in the manner as defined in SEQ ID NO:4. More preferably, the peptide comprising an amino acid sequence that has at least 90% sequence identity to the amino acid sequence of SEQ ID NO:4 has all 7 hydroxylated amino acid residues as defined in SEQ ID NO:4, and therefore the same hydroxylation pattern as defined in SEQ ID NO:4.

The peptide of SEQ ID NO:1 is hydroxylated at six position, i.e. position 15 (proline), position 17 (proline), position 18 (proline), position 24 (proline), position 27 (proline) and position 30 (lysine). Preferably, at position 15, the proline is a 4-hydroxyproline. Preferably, at position 17, the proline is a 3-hydroxyproline. Preferably, at position 18, the proline is a 4-hydroxyproline. Preferably, at position 24, the proline is a 4-hydroxyproline. Preferably, at position 27, the proline is a 4-hydroxyproline. Preferably, at position 30, the lysine is a 5-hydroxylysine. Most preferably, the peptide of SEQ ID NO:1 has at position 15 a 4-hydroxyproline; at position 17 a 3-hydroxyproline; at position 18 a 4-hydroxyproline; at position 24 a 4-hydroxyproline; at position 27 a 4-hydroxyproline; and at position 30 a 5-hydroxylysine.

The peptide of SEQ ID NO:2 is hydroxylated at four positions, i.e. position 8 (lysine), position 9 (proline), position 15 (proline) and position 21 (proline). Preferably, at position 8, the lysine is a 5-hydroxylysine. Preferably, at position 9, the proline is a 4-hydroxyproline. Preferably, at position 15, the proline is a 4-hydroxyproline. Preferably, at position 21, the proline is a 4-hydroxyproline. Most preferably, the peptide of SEQ ID NO:2 has at position 8 a 5-hydroxylysine; at position 9 a 4-hydroxyproline; at position 15 a 4-hydroxyproline; and at position 21 a 4-hydroxyproline.

The peptide of SEQ ID NO:4 is hydroxylated at seven positions, i.e. position 6 (proline), position 9 (proline), position 15 (proline), position 21 (proline), position 24 (proline), position 33 (proline) and position 35 (proline). Preferably, one or more of the hydroxylated prolines at positions 6, 9, 15, 21, 24, 33 and 35 is a 4-hydroxyproline (4Hyp). More preferably, all hydroxylated prolines at positions 6, 9, 15, 21, 24, 33 and 35 are 4-hydroxyproline (4Hyp).

The term “% sequence identity” is defined herein as the percentage of amino acids in an amino acid sequence that is identical with the amino acids in an amino acid sequence of interest, after aligning the sequences and optionally introducing gaps, if necessary, to achieve the maximum percent sequence identity. Methods and computer programs for alignments are well known in the art. Sequence identity is calculated over substantially the whole length, preferably the whole (full) length, of an amino acid sequence of interest. The skilled person understands that consecutive amino acid residues in one amino acid sequence are compared to consecutive amino acid residues in another amino acid sequence. The term “% sequence identity”, as used herein, requires that a hydroxylated amino acid residue at a certain position in the reference sequence (i.e. in SEQ ID NO:1, SEQ ID NO:2 or SEQ ID NO:4) is only considered identical to a target amino acid residue in a target amino acid sequence if the target amino acid residue is also hydroxylated at that position.

The skilled person has ample well known methods and means at his disposal for measuring peptide or protein levels in a sample, including measurement of relative or absolute peptide or protein concentrations, and/or longitudinal (multiple sampling of the same patient over time) or cross-sectional (a single time point measurement per patient) measurements.

Exemplary methods for peptide or protein analysis include, but are expressly not limited to, High-performance liquid chromatography (HPLC); mass spectrometry (MS), preferably set up in MS/MS mode; LC-MS based peptide profiling, preferably HPLC-MS, preferable set up in MS/MS mode (shotgun mode/data dependent acquisition (DDA), data independent acquisition (DIA), targeted mode (selected reaction monitoring (SRM), parallel reaction monitoring (PRM) and multiple reaction monitoring (MRM)) and the like. Preferably, PRM is employed. In the present invention, the methods provide for a quantitative detection of whether the peptide or protein is present in the sample being assayed, i.e., an evaluation or assessment of the actual amount or relative abundance of the peptide or protein in the sample being assayed. In such embodiments, the quantitative detection may be absolute or relative. As such, the term “level” or “quantifying” when used in the context of quantifying, or measuring a peptide or protein level in a sample can refer to absolute or to relative quantification. Absolute quantification may be accomplished by inclusion of known concentration(s) of one or more control analytes and referencing the detected level of the target peptide or protein with the known control analytes (e.g., through generation of a standard curve). Alternatively, relative quantification can be accomplished by comparison of detected levels or amounts between two or more different target peptides or proteins to provide a relative quantification of each of the two or more different peptides or proteins, e.g., relative to each other. In addition, a relative quantitation may be ascertained using a control, or reference, value (or profile) from one or more control or reference sample(s).

The fragmentation of a hydroxylated peptide by e.g. MS-MS identifies the position of the hydroxyl group, i.e. the hydroxylation pattern. Other suitable methods to determine the hydroxylation pattern are any method that measures the interaction of any of the hydroxylated peptides, such as immuno assays, multiplex assays, competitive assays, beads, carrier chips, arrays, sticks, columns. A suitable method may be immunoassay, multiplex assay, competitive assay and selection reaction monitoring (SRM). The detection may be indicated by any suitable means available such as chemiluminescence and/or fluorescence.

When MS is employed as peptide measurement tool, these peptide sequences provides for the benefit that peaks in the generated MS profile corresponding to these peptides can be easily identified and attributed to a hydroxylated NOP peptide biomarker as described herein. MS peaks of such a peptide is a measure for its peptide level. It should however be understood that peptide levels can be measured by numerous other methods.

It is within the routine capabilities of the skilled person, in a method for typing as described herein, to type said subject for the presence or absence of said secondary liver cancer on the basis of the measured peptide level. It follows for instance from the present application that the peptide level of a peptide as described herein is increased in samples of subjects that suffer from a secondary liver cancer as compared to healthy individuals. This knowledge allows the skilled person to set the threshold levels it deems appropriate.

A method for typing of the invention may further comprise a step of comparing the measured peptide level to a reference peptide level for said (i) peptide comprising the amino acid sequence of SEQ ID NO:1. SEQ ID NO:2 or SEQ ID NO:4, or (ii) peptide comprising an amino acid sequence that has at least 90% sequence identity with the amino acid sequence of SEQ ID NO:1, SEQ ID NO:2 or SEQ ID NO:4.

After measuring the peptide level of the target peptide, and for instance providing such peptide level data in the form of a profile or signature, the peptide level is analyzed or evaluated to determine whether the subject as described is typed as having, or not having, a secondary liver cancer. Such an analysis involves comparison of the measured peptide level to a reference peptide level for the same peptide.

The term “reference peptide level” denotes a standardized peptide level (or standardized peptide level profile or signature, or total normalized peptide level) that can be used to interpret the peptide level measured in a sample of a subject as described herein.

A reference peptide level that is appropriate for typing purposes of the present invention can be set by a skilled person in multiple, alternatives ways, such setting of reference peptide levels belonging to common general knowledge of the skilled person. For instance, in a method for typing of the invention, a reference peptide level can be a reference peptide level of said peptide in a reference sample, preferably obtained on the basis of a reference sample. The reference sample can be a sample from any individual, such as a healthy or diseased individual, but is preferably a sample from a healthy subject, preferably a healthy human subject. Such a sample can for instance be a (urine) sample of a healthy kidney donor, preferably obtained before organ donation. Such a sample can be a sample from a healthy subject not suffering from cancer (such as colorectal cancer) and not having suffered from cancer (such as colorectal cancer). Alternatively, such a sample can be a sample from a subject that is suffering from, or has suffered from, a primary cancer, preferably a primary colorectal cancer, which primary cancer has not (yet) developed into a secondary liver cancer. A peptide level of SEQ ID NO:1, SEQ ID NO:2 or SEQ ID NO:4 in subjects suffering from secondary liver cancer is preferably increased as compared to subjects suffering from a primary cancer, preferably a primary colorectal cancer, that has not (yet) developed into a secondary liver cancer.

Knowing the peptide level direction that is associated with secondary liver cancer, the skilled person can perform a method for typing as described herein by routinely applying appropriate reference peptide levels that either represent similarity or dissimilarity to secondary liver cancer peptides levels. Preferably, in a method for typing as described herein, when said measured peptide level is increased as compared to a reference peptide level for said peptide (wherein said reference peptide level is of a healthy subject), said subject, or said sample thereof, is typed as having secondary liver cancer present. Alternatively, when said measured peptide level is decreased as compared to, or equal to, a reference peptide level for said peptide (wherein said reference peptide level is of a healthy subject), said subject, or said sample thereof, is typed as not having secondary liver cancer (i.e. absent).

The reference sample can also be a pooled peptide sample from multiple individuals, such as healthy individuals as described above. Said sample can be pooled from more than 10 individuals, more than 20 individuals, more than 30 individuals, more than 40 individuals or more than 50 individuals.

Another beneficial reference peptide level is an absolute peptide level for discriminating secondary liver cancer from non-secondary liver cancer. It is within the common knowledge of the skilled person to set such an absolute threshold protein level.

Typing of a subject in a method for typing of the invention can be performed in multiple ways. In one method, a coefficient is determined that is a measure of a similarity or dissimilarity to the peptide level in a target sample, i.e. the sample that is to be investigated. Typing of a subject or sample can be based on its (dis)similarity to a single reference profile template or multiple reference profile templates. By determining a correlation with a profile template an overall similarity score can be set. A similarity score is a measure of the average correlation of a peptide level in a sample from a subject and a reference profile template. Said similarity score can, but does not need to be, a numerical value between +1, indicative of a high correlation between the peptide level and said profile template, and −1, which is indicative of an inverse correlation. A threshold value can then be set to differentiate between samples that are to be typed as secondary liver cancer or non-secondary liver cancer. Said threshold is an arbitrary value that allows for discrimination between secondary liver cancer or non-secondary liver cancer samples. If a similarity threshold value is employed, it is preferably set at a value at which an acceptable number of subject with secondary liver cancer would score as false negatives, and an acceptable number of subjects without secondary liver cancer would score as false positives. A similarity score is preferably displayed or outputted to a user interface device, a computer readable storage medium, or a local or remote computer system.

A classic method for calculating a similarity score when having different predictors is linear logistic regression, but there are further statistical and data mining classification methods available to the skilled person that can be used to calculate similarity scores. For instance, a non-limiting example is a support vector machine, which is a statistical learning method for building classification models (Cristianini et al., An Introduction to Support Vector Machines and Other Kernel-based Learning Methods., 2000, Cambridge University Press; Vapnik, The Nature of Statistical Learning Theory., 1995 New York Springer; Zhang et al., BMC Bioinformatics. 7:197 (2006)).

The method for typing according to the invention may further comprise the step of:—measuring in a sample comprising proteins from said subject a carcinoembryonic antigen (CEA) protein level;—typing said subject for the presence or absence of a secondary liver cancer on the basis of the measured peptide level and the measured CEA protein level.

Unexpectedly, it was established that the predictive power of method for typing of the invention could be substantially increased when a combination is used of (i) a measured peptide level of a peptide as described herein and (ii) blood CEA protein levels.

CEA is a protein that is normally not detected in the blood of a healthy individual. CEA is produced by certain cancer types and is often used to monitor patients with cancers of the gastrointestinal (GI) tract, such as colorectal cancer, to screen for development of secondary liver cancer in the period after resection of the primary tumor.

The skilled person is well aware of suitable methods and means for measuring CEA protein levels in relation to secondary liver cancer. The skilled person can employ MS-based protein measurement techniques as described hereinabove in relation to peptide level measurements. In addition, the skilled person can employ standard immunoassays available for clinical use including antibody- or aptamer-based protein quantification assays (e.g., enzyme-linked immunosorbent assay (ELISA) assays, such as a multiplex or sandwich ELISA assay, Western blots, FACS-based protein analysis, and the like). Commercial kits for assaying CEA protein levels are generally available. For instance, Abcam, plc. sells ‘Human Carcinoembryonic Antigen ELISA Kit (CD66e) (ab183365)’, which is an kit for a quantitative sandwich ELISA assay. This assay allows for measuring CEA protein levels in blood samples, such as serum or plasma samples.

Preferably, when measuring CEA protein levels, the sample of a subject is a blood sample, more preferably a serum sample. The skilled person understands that, in a method for typing according to the invention, two samples of a subject can be obtained, such as a first urine sample in order to measure a peptide level of a peptide as described herein, and a second blood sample in order to measure a CEA protein level.

In a method for typing of the invention, wherein peptide levels of peptides as described herein are measured and CEA protein levels are measured, the probability of having secondary liver cancer can be calculated by using a variety of formulas of which one optional and non-limiting example is shown herein below:

$$Probability of having secondary liver cancer = \frac{1}{1 + {e}^{- 1 * ( - 24.1476 + 3.0365 * GND + 3.4647 * CEA )}}$$

wherein “GND” is the measured peptide level of the peptide of SEQ ID NO:1, or of a peptide comprising an amino acid sequence that has at least 90% sequence identity to the amino acid sequence of SEQ ID NO:1, and wherein “GND” is expressed in area under the peak (curve) as measured by mass spectrometry; and “CEA” is the measured CEA protein level expressed in ng CEA/ml serum. For completeness sake, this non-limiting exemplary formula reads as 1/(1+e^{-1×(-24.1476:3.0365×GND+3.4647×CEA)}). The formula gives as output a value between 0 and 1. It is within routine capabilities of the skilled person to set a threshold or cut-off value between 0 and 1 that allows for distinguishing between healthy and diseased subjects. One suitable threshold or cut-off value that can be used to distinguish between healthy and diseased subjects is 0.439. Samples that score below 0.439 are regarded healthy, and samples that score above 0.439 are regarded diseased. Again, the skilled person has numerous alternative routine methods and means at his disposal to calculate such a probability value. In the same manner, when a peptide level of a peptide of SEQ ID NO:4 or of a peptide comprising an amino acid sequence that has at least 90% sequence identity to the amino acid sequence of SEQ ID NO:4 is measured, and CEA protein levels are measured, the probability of having secondary liver cancer can be calculated by using a variety of formulas of which one optional and non-limiting example is shown herein below:

$$Probability of having secondary liver cancer = \frac{1}{1 + {e}^{- 1 * ( - 20.62 + 3.05 * CEA + 2.49 * GER )}}$$

wherein “GER” is the measured peptide level of the peptide of SEQ ID NO:4, or of a peptide comprising an amino acid sequence that has at least 90% sequence identity to the amino acid sequence of SEQ ID NO:4, and wherein “GER” is expressed in area under the peak (curve) as measured by mass spectrometry; and “CEA” is the measured CEA protein level expressed in ng CEA/ml serum. For completeness sake, this non-limiting exemplary formula reads as 1/(1+e^{-1×(-20.62+3.05×CEA+2.49×GER)}).

A method for typing of the invention may further comprise the steps of—comparing said measured protein level to a reference CEA protein level; and—typing said subject for the presence or absence of a secondary liver cancer on the basis of the (i) comparison of the measured peptide level and the reference peptide level and (ii) comparison of the measured CEA protein level and the reference CEA protein level.

Appropriate reference CEA protein levels can be set in the same manner as described above in relation to reference peptide levels. Said reference CEA protein level can be measured in a sample comprising proteins from a reference subject that is a healthy individual. Said reference CEA protein level can be measured in a sample comprising proteins from a reference subject that is not suffering from, or has not suffered from cancer, including colorectal cancer.

The invention also provides a use of a (i) peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4, in typing a subject for the presence or absence of a secondary liver cancer. The invention also provides a use of a peptide level of a peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4, in typing a subject for the presence or absence of a secondary liver cancer. Preferably, such a use is the combined use of (i) the peptide or peptide levels as described above with (ii) CEA or CEA protein levels. Embodiments described above in relation to a method for typing are also disclosed in relation to a use of the invention. For instance, the peptide in said use is preferably a peptide as described herein.

The invention also relates to a peptide as defined in relation to a method for typing of the invention, including a peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4. Preferably, the sequence identity is at least 91%, 92%, 93%, 94%, 95%, 97%, 98% or at least 99%. Preferably, the peptide comprising an amino acid sequence that has at least 90% sequence identity to the amino acid sequence of SEQ ID NO:1 has at least 1, 2, 3, 4, 5 or 6 hydroxylated amino acid residues in the manner as defined in SEQ ID NO:1. More preferably, the peptide comprising an amino acid sequence that has at least 90% sequence identity to the amino acid sequence of SEQ ID NO:1 has all 6 hydroxylated amino acid residues as defined in SEQ ID NO:1, and therefore the same hydroxylation pattern as defined in SEQ ID NO:1. Preferably, the peptide comprising an amino acid sequence that has at least 90% sequence identity to the amino acid sequence of SEQ ID NO:2 has at least 1, 2, 3 or 4 hydroxylated amino acid residues in the manner as defined in SEQ ID NO:2. More preferably, the peptide comprising an amino acid sequence that has at least 90% sequence identity to the amino acid sequence of SEQ ID NO:2 has all 4 hydroxylated amino acid residues as defined in SEQ ID NO:2, and therefore has the same hydroxylation pattern as defined in SEQ ID NO:2. Preferably, the peptide comprising an amino acid sequence that has at least 90% sequence identity to the amino acid sequence of SEQ ID NO:4 has at least 1, 2, 3, 4, 5, 6 or 7 hydroxylated amino acid residues in the manner as defined in SEQ ID NO:4. More preferably, the peptide comprising an amino acid sequence that has at least 90% sequence identity to the amino acid sequence of SEQ ID NO:4 has all 7 hydroxylated amino acid residues as defined in SEQ ID NO:4, and therefore the same hydroxylation pattern as defined in SEQ ID NO:4.

Preferably, the peptide is an isolated peptide. The peptide is preferably at least partially purified, and may have a purity of at least 40%, 50%, 60%, 70%, 80%, 90%, 91%, 92%, 93%. 94% or at least 95%. The peptide can also be a chemically synthesized peptide, optionally comprising a label such as a fluorescent or a stable isotope label (SIL).

The invention further provides medical methods, including a standard-of-care therapeutic agent against a secondary liver cancer for use in treating a subject typed as having a secondary liver cancer according to the method for typing as defined above. Preferably, the secondary liver cancer is CRLM. Embodiments disclosed in relation to a method for typing that also apply to the present medical use are disclosed in relation to this medical use.

In the same manner, the invention provides a use of a standard-of-care therapeutic agent against a secondary liver cancer for the manufacture of medicament for treating a subject suffering from a secondary liver cancer; wherein said subject is typed as having a secondary liver cancer according to a method for typing according to the invention. Preferably, the secondary liver cancer is CRLM. Embodiments disclosed in relation to a method for typing that also apply to the present medical use are disclosed in relation to this medical use.

The invention also provides a method for treating a subject suffering from a secondary liver cancer, comprising the step of—performing a method for typing of the invention; and
administering a therapeutically effective amount of a standard-of-care therapeutic agent against secondary liver cancer when said subject is typed as having a secondary liver cancer. Preferably, the secondary liver cancer is CRLM. Embodiments disclosed in relation to a method for typing that also apply to the present medical use are disclosed in relation to this medical use.

The term “standard-of-care therapeutic agent”, as used herein, refers to a therapeutic compound, or a combination of such compounds, that is/are considered by medical practitioners as appropriate, accepted, and/or widely used for a certain type of patient, disease or clinical circumstance that is secondary liver cancer. Standard-of-care therapies for counteracting secondary liver cancer are available in the art. Standard-of-care therapeutic agents for use in treating secondary liver cancer include a targeted agent such as an antibody including cetuximab; bevacizumab; or panitumumab. Another targeted agent is aflibercept. Specific standard-of-care therapeutic agents for use in treating secondary liver cancer also include one or more chemotherapeutic agents such as FOLFOX (folinic acid, fluorouracil, and oxaliplatin) or FOLFIRI (folinic acid, fluorouracil and irinotecan).

The term “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic agent. It is within the knowledge and capabilities of the skilled practitioner to determine therapeutically effective dosing regimens.

The term “administering”, as used herein, refers to the physical introduction of an agent or therapeutic compound to a subject suffering from a secondary liver cancer, using any of the various methods and delivery systems known to those skilled in the art. The skilled person is aware of suitable methods for administration and dosage forms. Administration of small molecules can generally be performed by non-parenteral administration such as by oral and enteral administration. Preferred route of administration for protein-based agents such as antibodies is by parenteral administration, including intravenous, intramuscular, subcutaneous, intraperitoneal, spinal or other parenteral routes of administration, executed inter alia by injection or infusion in the form of a solution. Administering can be performed, for example, once, a plurality of times, and/or over one or more extended periods of time.

The invention also provides a method for measuring a peptide level, comprising the step of:—optionally, providing a sample comprising peptides from a subject;—measuring in a sample comprising peptides from a subject a peptide level for (i) a peptide comprising the amino acid sequence of SEQ ID NO:1, SEQ ID NO:2 or SEQ ID NO:4 or (ii) a peptide comprising an amino acid sequence that has at least 90% sequence identity to the amino acid sequence of SEQ ID NO:1, SEQ ID NO:2 or SEQ ID NO:4. Preferably the method for measuring a peptide level also comprises a step of—optionally, providing a sample comprising proteins from said subject;—measuring in a sample comprising proteins from said subject a carcinoembryonic antigen (CEA) protein level. Preferably, said sample comprising peptides is a urine sample and said sample comprising proteins is a blood sample, preferably a serum or plasma sample.

Embodiments described in this text in relation to the steps of providing a sample and measuring peptide or protein levels, are also embodiments in a method for measuring a peptide (and protein) level as described herein.

For the purpose of clarity and a concise description, features are described herein as part of the same or separate embodiments, however, it will be appreciated that the disclosure includes embodiments having combinations of all or some of the features described.

The content of the documents referred to herein is incorporated by reference.

### FIGURE LEGENDS

FIG. 1. Flowchart study

FIG. 1 shows a flowchart of the samples used in this study, showing discovery cohort 1 and validation cohort 2.

FIG. 2. Optimized collision energies NOPs

FIG. 2 lists, among other characteristics, the optimized collision energies of NOPs AGP, GPP and GND.

FIG. 3. Scatterplot optimal LRM (GND+CEA) and old LRM (AGP+CEA)

The scatter plot shows prediction of CRLM using the new combination of biomarkers (GND+CEA; optimal LRM) (left half of scatterplot) and a known combination of biomarkers (AGP+CEA; old LRM) (right half of scatterplot). The striped line represents the optimal cut-off for each model.

SEQUENCE LISTING
SEQ ID NO: 1: Hydroxylated GND peptide
GNDGARGSDGQPGPP(—OH)GP(—OH)P(—OH)GTAGFP(—OH)
GSP(—OH)GAK(—OH)GEVGP
SEQ ID NO: 2: Hydroxylated GPP peptide
GPPGEAGK(—OH)P(—OH)GEQGVP(—OH)GDLGAP(—OH)GP
SEQ ID NO: 3: Hydroxylated AGP peptide
AGPP(—OH)GEAGKP(—OH)GEQGVP(—OH)GDLGAP(—OH)GP
SEQ ID NO: 4: Hydroxylated GER peptide
GERGSP(—OH)GGP(—OH)GAAGFP(—OH)GARGLP(—OH)GPP
(—OH)GSNGNPGPP(—OH)GP(—OH).

In the SEQ ID NOs, (—OH) indicates that the preceding amino acid residue is hydroxylated. As an example, P(—OH)G means that P is hydroxylated.

### EXAMPLES

### Example 1

Materials and Methods

Experimental Design and Statistical Rationale

This study was approved by the Erasmus MC ethics review board (MEC-2008-062) and was performed according to the declaration of Helsinki. Urine samples of healthy kidney donors (controls) and CRLM patients were measured alternately with mass spectrometry.

The identification of new collagen NOPs in urine was based on the identification of all NOPs in urine (discovery set 1: controls, n=40; CRLM, n=40). In a previous study, a sample size of 25 samples per group proved sufficient to identify peptide based markers in bottom-up proteomics in tissue (Van Huizen et al., J Biol Chem. 294:281-9 (2018)). However, in urine the observed differences in NOP levels are smaller. The mean and standard deviations (SD) used for the power analysis (alpha=0.05, beta=0.20) were calculated from the overall data of log-transformed significant upregulated collagen peptides in urine samples of five CRLM patients and five control patients (control mean=6.76, CRLM mean=6.98, SDpooled=0.75). The power analysis resulted in a sample size of 40 samples per group.

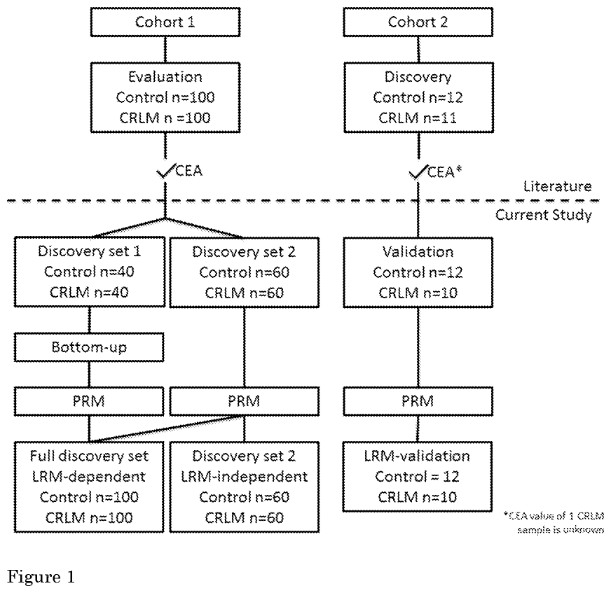

Targeted analysis on NOPs of interest was performed on discovery set 1 and on an additional urine sample set (discovery set 2: control, n=60; CRLM, n=60). The discovery sets 1 and 2 as used herein for discovery are described in Lalmahomed et al., Am J Cancer Res., 6:321-30 (2016). Validation was performed on independently collected urine samples (control, n=12; CRLM, n=10) (Broker et al., Plos One; 8:e70918 (2013)). A flow chart of the samples used is shown in FIG. 1.

Bottom-up proteomics was used to identify new NOPs. Assessment of the number of significant NOPs by chance in the bottom-up proteomics data was determined by permutation testing.

The three most significant NOPs associated with the three most abundant collagen alpha chains that are also more strongly upregulated in CRLM tissue than in healthy liver tissue were selected (Van Huizen et al., J Biol Chem. 294:281-9 (2018)) As bottom-up proteomics is a semi-quantitative technique, a targeted quantitative mass spectrometry method (parallel reaction monitoring, PRM) was developed to validate these findings.

The developed PRM method is conform tier 3 (of 3 levels) of analytical assay validation (Carr et al., Mol Cell Proteomics; 13:907-17(2014)), which implies that the assay is a targeted discovery assay. The PRM method was applied on both the full discovery set and the validation set. To determine the best model, a logistic regression model (LRM) was fit, containing the NOPs (referred to by the three letter code) and CEA. The optimal LRM was fit by backward elimination of predictors from the LRM that contained all molecular markers (AGP, GND, GPP, and CEA). The optimal model was validated on the validation set. The statistical analysis was performed on the discovery set 2, and on the combined discovery sets 1 and 2 (full discovery set). However, combining the discovery set 1 with discovery set 2 created a dependent data set, because discovery set 1 was already used for bottom-up proteomics. Yet, combining generates a higher statistical power. After measuring all samples, and optimizing the LRM, we selected three samples with low, medium, and high levels of the predictors present in the optimal-LRM. Because SIL peptides were not available for all predictors, these three samples were processed five times to get an estimate of the reproducibility.

Chemicals

Ultra-high pressure liquid chromatography grade solvents were obtained from Biosolve (Valkenswaard, the Netherlands). A stable isotope labeled (SIL) peptide was obtained for AGPP(—OH)GEAGK(SIL)P(—OH)GEQGVP(—OH)GDLGAP(—OH)GP from Pepscan (Lelystad, the Netherlands), the lysine is labelled with ¹³C₆¹⁵N₂. This SIL peptide was characterized using HPLC-UV and ESI-MS. Other peptides are GPPGEAGK(—OH)P(—OH)GEQGVP(—OH)GDLGAP(—OH)GP and GNDGARGSDGQPGPP(—OH)GP(—OH)P(—OH)GTAGFP(—OH)GSP(—OH)GAK(—OH)GEVGP.

These three urine NOPs will be abbreviated by the first three amino acids (AGP, GPP, and GND, respectively).

All other chemicals were obtained from Sigma-Aldrich (Zwijndrecht, the Netherlands).

Sample Selection

Samples of the cohorts described in the studies of Lalmahomed et al., Am J Cancer Res., 6:321-30 (2016) and Broker et al., Plos One; 8:e70918 (2013) were reanalyzed (FIG. 1). Samples of cohorts 1 and 2, were, after collection, stored at −80° C. in polypropylene tubes. One CRLM sample was excluded from the validation set of the current study because the corresponding CEA value was not known. As CEA levels for the validation set of the Broker et al., 2013 study are not known, this set of samples was excluded.

Age and BMI differences between controls and CRLM patients were calculated with a t-test, and differences in gender and serum creatinine levels above 115 μM/L with a chi-square test. A p-value below 0.05/4=0.0125 (Bonferroni correction to correct for multiple testing) was considered as significant.

Sample Preparation

NOPs for bottom-up proteomics and targeted mass spectrometry were isolated from urine as described by Lalmahomed et al., Am J Cancer Res., 6:321-30 (2016). In brief, NOPs were separated from small molecules, salts, and proteins with a mRP C-18 Hi-Recovery Protein Column (4.6×50 mm) (Agilent, Amstelveen, the Netherlands) installed in an Ultimate 300 LC system (Dionex, Amsterdam, the Netherlands) equipped with an online-fractionator. After separation, the NOP fraction was collected, dried, reconstituted, and analyzed with mass spectrometry.

Bottom-Up Proteomics

For the identification of NOPs we applied a standard bottom-up LC-MS/MS method as described Van Huizen et al., J Biol Chem. 294:281-9 (2018). In short, an Ultimate 3000 nano RSLC system (Thermo Fischer Scientific, Germering, Germany) was coupled online to an Orbitrap Fusion Lumos Tribrid Mass Spectrometer (Thermo Fischer Scientific, San Jose, Calif., USA). Injected samples were trapped and washed on a trap column (C18 PepMap, 300 μm ID×5 mm, 2 μm particles size, 100 Å pore size; Thermo Fisher Scientific, the Netherlands). After washing, the trap column was switched in line with an analytical column (PepMap C18, 75 μm ID×250 mm, 2 μm particle size, 100 Å pore size; Thermo Fisher Scientific, the Netherlands) for peptide separation prior to mass spectrometry analysis. We deviated from the protocol described by van Huizen et al., 2018 in that prior to mass spectrometry analysis, neither the samples were analyzed on a test HPLC-system, nor the injection volume was normalized. For every sample, a volume of 2 μL was injected. Bottom-up proteomics data was uploaded to the PRIDE archive (PXD013533).

Analysis of Bottom-Up Data

MGF peak list files were extracted from raw files by ProteoWizard (v3.0.9166). MGF peak list files were searched using the Mascot search engine (v2.3.2, Matrix Science Inc., London, UK) and the UniProt/SwissProt database (20194 entries). The following settings were used for the database search: enzyme was set to open because we analyzed NOPs; the mass tolerance was set to 10 ppm for peptide mass and 0.5 Da for fragment mass. As variable modification hydroxylation of proline, lysine, and oxidation of methionine was selected (+16 Da); no fixed modifications were added. MASCOT identifications were imported into Scaffold (v4.6.2, Portland, Oreg., USA). In Scaffold, protein confidence levels were set to 1% false discovery rate (FDR), at least 2 peptides per protein, and a 1% FDR at peptide level. FDRs were estimated by inclusion of a decoy database search generated by MASCOT. Raw files were aligned and combined with the identification list exported from Scaffold in Progenesis QI (v4, Nonlinear Dynamics, Newcastle-upon-Tyne, United Kingdom) followed by exporting the normalized abundance to Excel 2010 (Microsoft, Redmon, Wash., USA). Duplicate feature intensities were summed. Data was further processed with Excel, GraphPad Prism (v5.01, La Jolla, Calif., USA), and R (v3.3.1, Vienna, Austria). Prior to ¹⁰ log-transformation, a value of ‘10’ was added, with the aim to include missing values for further data analysis. After ¹⁰ log-transformation the data was assumed to be normally distributed. With an unequal variance independent samples t-test NOP significance between control and CRLM was tested. P-values below 0.05 were considered significant.

Only collagen alpha chains were taken into account, which also were found to differ between CRLM tissue and normal liver tissue (Van Huizen et al., J Biol Chem. 294:281-9 (2018)). A NOP molecular panel was constructed consisting of nine NOPs, i.e., the three most significant NOPs from the top three most abundant collagen alpha chains. In addition to these nine NOPs, the earlier reported NOP named AGP (Lalmahomed et al., Am J Cancer Res., 6:321-30 (2016); Broker et al., Plos One; 8:e70918 (2013)), was included in the targeted mass spectrometry method.

Permutation testing was performed according to the R-script published as supplemental file by Van Huizen et al., J Biol Chem. 294:281-9 (2018). In short, the data was randomly divided in two groups at the peptide level; significant differences between the two groups were determined using the Wilcoxon signed-rank test. Significant differences (p-value <0.05) were summed per permutation and the ¹⁰ log was taken. The distribution of the ¹⁰ log summed significant p-values was assumed to be normal. The difference was assumed to be significant if the true dataset value was greater than the average value of the permutation test plus twice the SD (p<0.05).

Targeted Mass Spectrometry Analysis

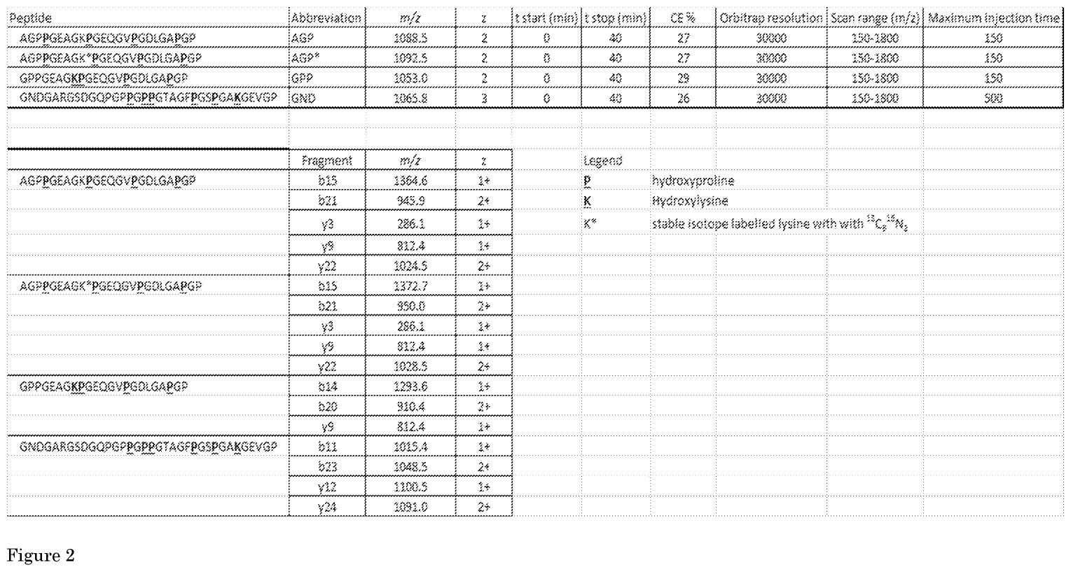

Targeted mass spectrometry measurements were performed on the same nanoLC-ESI-Orbitrap Lumos Fusion as used for the bottom-up proteomics. To measure the samples, a PRM method with optimized collision energies was developed. NOPs for which no optimal collision energy could be determined or which had too low signal intensities for identification were excluded. A table listing, among other characteristics, the optimized collision energies is available in FIG. 2.

The full discovery set and the validation set were measured at different times to increase validity. The data sets were aligned to the discovery set 1 using the mean values of the control groups of the other data sets. This was only necessary for NOPs for which no SIL peptides were generated. Targeted mass spectrometry data was uploaded to the PRIDE archive (PXD013705).

Analysis of Targeted Data

Raw files produced by the mass spectrometer were imported into Skyline (MacLean et al., Bioinformatics; 26:966-8 9 (2010)). Per peptide, we selected a maximum of five transitions with a high intensity, and no obvious interference of neighboring peaks. The GND and GPP peptide peak areas from Skyline were used and for AGP a ratio with the SIL peptide was used.

Logistic Regression Model

Statistical analyses were performed in R (version 3.3.1, Vienna, Austria) (R Core Team, R Foundation for Statistical Computing, Vienna, Austria. Retrieved from https://www.R-project.org/. (2016)). The predictor selection was applied separately on the discovery set 2 (independent data set) and the full discovery set (dependent data set). If the data set (full discovery set or discovery set 2) used to select predictors did not show a different predictor selection, than the analysis was performed with the full discovery set to prevent a loss of power. To select relevant predictors to fit the optimal logistic regression model, a significance level of 0.05 was used. The critical p-value was Bonferroni corrected for the number of predictors or comparisons tested.

The current molecular panel consists of AGP and CEA, which was extended with the newly identified NOPs (GPP and GND). To fit a new model with the molecular markers, these markers were tested on any relationship between patient characteristics, individual significance, and multicollinearity. A relationship between the patient characteristics ‘age’, ‘gender’, ‘BMI, ‘serum creatinine >115 μM/L’ was determined by fitting a linear model that predicts an individual molecular marker per patient characteristic and the predictor ‘group (healthy/sick)’. Molecular markers that were significantly correlated with a patient characteristic were excluded from further analysis. All remaining individual predictors were tested for significance by fitting a LRM with the individual predictors. Significance of an individual predictor was based on Wald statistics. The selected significant predictors were assessed for multicollinearity by calculating the variance inflation factor (VIF). Multicollinearity was assumed to be present with a VIF above 10; if necessary predictors were discarded to prevent multicollinearity.

The selected predictors were fit into a combined LRM (full-LRM). The optimal LRM (optimal-LRM) was formed by backward elimination of non-significant predictors from the full-LRM.

The relation between the molecular markers in the optimal-LRM and the size of the largest tumor, as well as, the number of tumors were tested by fitting a linear model. Significance of an individual predictor was based on Wald statistics.

The Cook's distance test was used to inspect the data for outliers and/or leverage points. The threshold for a point to be suspected of being an outlier/leverage point was calculated with the formula 4/(n−k−1), whereby n=number of samples, k=number of predictors. Outliers and/or leverage points identified by manual inspection of the samples were removed from the data set.

Our previous logistic regression model (old-LRM) contained AGP and CEA. Prior to comparison of the old-LRM and the optimal-LRM, a Pearson correlation between was calculated. To select the LRM with the highest predictive power, the performance of the optimal-LRM needed to be compared to that of the old-LRM. The predictive power was compared with the ‘anova’ function if there was nesting, otherwise the DeLong's test was used to compare AUCs.

Results

Patient Characteristics

Table 1 provides an overview of the basic patient characteristics. Age and gender were significantly different between the controls and CRLM patients. A serum creatinine level above 115 μM/L, indicating renal impairment, was measured in four patients.

TABLE 1
Patient characteristics

| | Full-Discovery | | | Validation | | |
|---|---|---|---|---|---|---|
| Parameter | Control | CRLM | p-value | Control | CRLM | p-value |
| Age (years) | 52 [43-63] | 64 [57-70] | <0.001 | 59 [54-65] | 66 [65-74] | 0.02 |
| Mate gender, No. (%) | 32 (32%) | 68 (68%) | <0.001 | 0 (0%) | 0 (0%) | — |
| BMI | 26 [23-28] | 26 [25-28] | 0.13 | 26 [23-28] | 25 [22-28] | 0.27 |
| No. of lesions | — | 2 [1-4] | — | — | 2 [1-2]¹ | — |
| Size of largest lesion (cm) | — | 2.7 [1.8-4] | — | — | 3.5 [3.0-4.5]¹ | — |
| Serum creatinine > 115 μM/L | 0 (0%) | 4 (4%) | 0.24 | 0 (0%) | 1 (10%) | 0.93 |

Data are presented as a median with the interquartile range (25th-75th percentile) or n (%)
¹For one patient the no. of lesions, and size of the largest lesion were not available.

Bottom-Up Mass Spectrometry

A total of 1683 NOPs were identified in the discovery set 1, belonging to 175 proteins. The three most common proteins are collagen type1(I) (n=183 NOPs), collagen type-1(III) (n=157 NOPs), and uromodulin (n=84 NOPs). Four hundred and fifty-three NOPs (27%) belong to 13 collagen alpha chains (Table 2). Four hundred and six NOPs (24%) were significantly different between control and CRLM, of which 118 belong to collagen (Table 2).

TABLE 2
Number of NOPs identified per collagen alpha chain.

| | | Up ^{II} | Down ^{II} |
|---|---|---|---|
| | | p < 0.05 & | p < 0.05 & |
| Protein ^{I} | Peptides | FC > 1 | FC < 1 |
| Collagen alpha-1(I) chain^{III} | 183 | 30 | 10 |
| Collagen alpha-2(I) chain^{III} | 54 | 8 | 2 |
| Collagen alpha-1(II) chain | 4 | 0 | 0 |
| Collagen alpha-1(III) chain^{III} | 157 | 56 | 1 |
| Collagen alpha-1(IV) chain | 3 | 1 | 0 |
| Collagen alpha-5(IV) chain | 4 | 1 | 0 |
| Collagen alpha-1(V) chain | 2 | 1 | 0 |
| Collagen alpha-2(V) chain | 8 | 3 | 0 |
| Collagen alpha-1(VI) chain | 3 | 0 | 0 |
| Collagen alpha-1(X) chain | 3 | 0 | 0 |
| Collagen alpha-1(XV) chain | 11 | 0 | 0 |
| Collagen alpha-1(XVIII) chain | 20 | 1 | 4 |
| Collagen alpha-1(XXII) chain | 1 | 0 | 0 |

^{I} Proteins marked in bold are shown to be upregulated in CRLM compared to normal liver tissue.
^{II} The number NOPs up- or downregulated with a p-value below 0.05 and the fold change (FC) above or below 1.
^{III}The three most abundant collagen alpha chains.

Targeted Mass Spectrometry

The urine NOP panel was constructed by including AGP (Lalmahomed et al., Am J Cancer Res., 6:321-30 (2016); Broker et al., Plos One; 8:e70918 (2013)) and the three most significantly different NOPs of the three most abundant collagen alpha chains. Optimal collision energy could not be determined for seven urine NOPs. The three remaining urine NOPs were AGPP(—OH)GEAGKP(—OH)GEQGVP(—OH)GDLGAP(—OH)GP, GPPGEAGK(—OH)P(—OH)GEQGVP(—OH)GDLGAP(—OH)GP, and GNDGARGSDGQPGPP(—OH)GP(—OH)P(—OH)GTAGFP(—OH)GSP(—OH)GAK(—OH)GEVGP. While AGP and GPP originate from collagen alpha chain 1(I), GND originates from collagen alpha chain 1(III).

Logistic Regression Model

The predictor selection process was applied on the discovery set 2 and the full discovery set. Prior to fitting the full-LRM, the molecular markers (AGP, GPP, GND, and CEA) were tested on a linear relationship with any of the patient characteristics (age, gender, BMI, and serum creatinine levels). Significant linear relationships were not found. The individual molecular markers were also tested for individual significance by fitting a LRM per marker. The results are shown in Table 3. Individually, all molecular markers showed to be significant and were included in the full-LRM. There was no multicollinearity present between the molecular markers. Therefore all molecular markers were included into the full-LRM in the full discovery set and in discovery set 2. In the full discovery set neither a significant linear relationship was present between any of the molecular markers individually, nor with any of the molecular markers and size of the largest tumor and number of tumors.

The optimal-LRM was formed with backwards elimination of the non-significant predictors. For both data sets, this resulted in a model containing GND and CEA (Optimal-LRM). The predictor selection was irrespective of the use of the full discovery set or discovery set 2. The remaining analyses were, therefore, performed solely with the full discovery set to prevent a loss of statistical power. The formula to predict the probability of an individual of having CRLM is shown in formula 1. The OR with 95% CI for GND is 21 [8.5-60] and for CEA 32 [10-129].

$$Formula 1$$

$$Probability of having secondary liver cancer = \frac{1}{1 + {e}^{- 1 * ( - 24.1476 + 3.0365 * GND + 3.4647 * CEA )}}$$

The Cook's distance was calculated to ensure that this formula is not heavily influenced by outliers/leverage points. Fourteen data points were above the threshold and were manually inspected. None appeared to be a wrong measurement, and therefore none was removed.

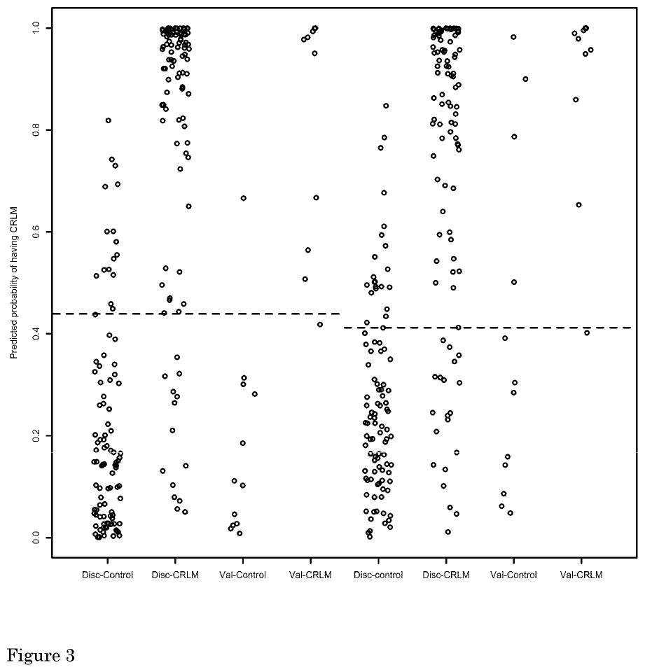

On re-measuring, AGP values from the old-LRM and the new optimal-LRM data sets were highly correlated (correlation=0.89, p-value <2.2*10⁻⁶). The linear relationship between the old AGP values (AGP_old) and the current AGP values is: AGP=0.9+1.57*AGP_old. The AUCs of the old-LRM and the optimal-LRM were compared using DeLong's test. The old-LRM had an AUC of 0.8824, which is significantly different from the optimal-LRM AUC of 0.9256 (p-value=0.032). A scatter plot containing the values of the optimal-LRM and old-LRM is available in FIG. 3.

Based on the ROC curve for values calculated by the optimal-LRM, a cut-off value of 0.439 was chosen. This cut-off value results in an 86% sensitivity and 84% specificity in the full discovery set, and in the validation set in 92% sensitivity and 90% specificity (Table 4).

To estimate the reproducibility of the sample processing with respect to the GND values, we measured three samples, selected in the lower, middle, and higher range of all measured values, five times. The following samples were measured, with in brackets the ¹⁰ log of the area and % CV: VMS-248 (low, 6.1±1.4%), VMS-253 (middle, 6.9±1.8%), and VMS-163 (high, 7.6±1.4%).

TABLE 3
Predictor selection, significant predictors are marked in bold

| | Univariable (Disc 2) | | Muitivariable (Disc 2) | | Univariable (Full disc) | | Multivariable (Full disc) | |
|---|---|---|---|---|---|---|---|---|
| Predictor | OR^{I} | p-value | OR^{I} | p-value | OR^{I} | p-value | OR^{I} | p-value |
| AGP | 1.7 [1.4-2.2] | 4.3 * 10⁻⁵ | 1.4 [1.0-2.0] | 0.11 | 1.6 [1.4-2.0] | 3.1 * 10−7 | 1.2 [0.9-1.5] | 0.27 |
| GPP | 4.0 [1.7-11.3] | 0.0039 | —^{III} | | 7.1 [3.1-18] | 1.3 * 10−5 | 0.7 [0.2-2.4] | 0.56 |
| GND | 211 [8.0-67] | 1.5 * 10−8 | 12 [3.4-46] | 1.2 * 10−4 | 20 [9.4-48] | 3.4 * 10−13 | 19 [6.1-66] | 1.3 * 10−6 |
| CEA | 12.7 [5.1-38] | 6.8 * 10−7 | 20 [5.4-104] | 6.2 * 10−5 | 21 [8.8-56] | 1.3 * 10−10 | 34 [11-146] | 8.3 * 10−8 |

^{I}The odds ratio (OR) is presented as the OR and the 95% confidence level
^{II}The critical p-value used is 0.05/15 = 3.3 * 10⁻³
^{III}This molecular marker was individually not significant, and therefore not selected for the full-LRM

TABLE 4
Overview of the GND and CEA values and the obtained sensitivity and specificity.

| | | | | Optimal-LRM | | Old-LRM | |
|---|---|---|---|---|---|---|---|
| | | GND^{I} | CEA^{I} | Sensitivity | Specificity | Sensitivity | Specificity |
| Discovery | Control | 6.9 [6.7-7.2] | 0.3 [0.1-0.5] | 86% | 84% | 80% | 79% |
| | CRLM | 7.7 [7.4-8.0] | 1.0 [0.6-1.4] | | | | |
| Validation | Control | 6.9 [6.7-7.1] | 0.2 [0.0-0.5] | 92% | 90% | 66% | 90% |
| | CRLM | 7.5 [7.3-7.6] | 1.5 [0.6-2.1] | | | | |

^{I}Mean [1^{st} quartile-3^{rd} quartile]

### Example 2

This Example is a supplement to Example 1.

Materials and Methods

The procedure as described in Example 1 was used to identify and test a further natural occurring peptide (NOP), also referred to as “GER”, with amino acid sequence GERGSP(−4Hyp)GGP(−4Hyp)GAAGFP(−4Hyp)GARGLP(−4Hyp)GPP(−4Hyp)GSNGNPGPP(−4Hyp)GP(−4Hyp) in urine. “P(−4Hyp)” means that the amino acid proline (P) is modified into 4-hydroxyproline. This natural occurring peptide (NOP) originates from collagen alpha-1(III) (COL3A1, protein code uniprot/Swissprot=P02461). A short description of this procedure is summarized below.

For the discovery of the novel NOP GER we had a large sample set of healthy control urine (n=100) and urine from patients suffering from CRLM (n=100) available. The sample set was split with a ratio 40:60. Identification of NOP GER as a novel marker for CRLM was based on the analysis of 40 controls and 40 CRLM urines using an unbiased semi-quantitative proteomics approach. Further validation of the value of NOP GER as such was performed by using a targeted quantitative mass spectrometry method on the full sample set (control n=100, CRLM n=100). NOP GND formed together with serum carcinoembryonic antigen (CEA) a panel of markers that were fit in a logistic regression model (LRM-GND). From LRM-GND, NOP GND was replaced with NOP GER (LRM-GER). First, it was tested if NOP GER had a significant contribution to the model based on the Wald-statistics (p-value ≤0.05) and the 95% confidence interval (CI) of the odds-ratio (not overlapping with 1). Second, the predictive power of both the LRM-GND and LRM-GER were compared by comparing the area under the curve (AUC) of the ROC-curve using DeLongs test, a p-value below 0.05 was considered significant.

Results

In the same manner as NOP GND, the NOP GER was identified as a biomarker for secondary liver cancer. Further, in Table 5, the results of the LRM-GER are displayed showing the significance of NOP GER in the model. NOP GER has a significant contribution to the model with a p-value of 3.60*10⁻⁷, which is confirmed by the 95% CI of the odds-ratio that does not overlap with 1.

TABLE 5
Significance of natural occurring peptide GER in a logistic regression
model to predict secondary liver cancer.

| | | | Odds | CI | CI |
|---|---|---|---|---|---|
| | Parameter | P-value | ratio | 2.5% | 97.5% |
| Intercept | −20.62 | 2.5e-09 | 0.00 | 0.00 | 0.00 |
| CEA | 3.05 | 1.5e-08 | 21.20 | 8.00 | 67.80 |
| COL3A1_GER | 2.49 | 2.3e-08 | 12.10 | 5.30 | 31.10 |

CI confidence interval
CEA carcinoembryonic antigen
COL3A1 collagen alpha-1(III)

The exemplary formula (Formula 2) that was used to calculate the chance for a patient of having secondary liver cancer based on LRM-GER was:

$$Probability of having secondary liver cancer = \frac{1}{1 + {e}^{- 1 * ( - 20.62 + 3.05 * CEA + 2.49 * GER )}}$$

The predictive power of LRM-GND and LRM-GER were compared based on the AUC of the ROC-curve. LRM-GER had a AUC of 0.9079 and LRM-GND of 0.9256. The AUCs are not significantly different (p=0.28), indicating that both models have a similar predictive power. Similar to the NOP GND, the combination of NOP GER and serum CEA has a significantly higher predictive power than serum CEA by itself and is similar to NOP GND in combination with CEA The standard for urine concentration correction is the level of creatinine in urine. Addition of urine creatinine levels to the models does not negatively influence the predictive powers of the NOP GND nor NOP GER (data not shown).

1. A method for typing a subject for the presence or absence of a secondary liver cancer, comprising the steps of
measuring in a sample comprising peptides from a subject a peptide level for
(i) a peptide comprising the amino acid sequence of SEQ ID NO:4 or a peptide comprising an amino acid sequence that has at least 90% sequence identity to the amino acid sequence of SEQ ID NO:4; and/or
(ii) a peptide comprising the amino acid sequence of SEQ ID NO:1 or a peptide comprising an amino acid sequence that has at least 90% sequence identity to the amino acid sequence of SEQ ID NO:1; and
typing said subject for the presence or absence of said secondary liver cancer on the basis of the measured peptide level.

2. The method according to claim 1, further comprising the steps of
comparing said measured peptide level to a reference peptide level for
(i) said peptide comprising the amino acid sequence of SEQ ID NO:4 or said peptide comprising an amino acid sequence that has at least 90% sequence identity to the amino acid sequence of SEQ ID NO:4; and/or
(ii) said peptide comprising the amino acid sequence of SEQ ID NO:1 or a peptide comprising an amino acid sequence that has at least 90% sequence identity to the amino acid sequence of SEQ ID NO:1; and
typing said subject for the presence or absence of a secondary liver cancer on the basis of the comparison of the measured peptide level and the reference peptide level.

3. The method according to claim 1, wherein the subject is a subject suffering from, or a subject having suffered from, a primary cancer.

4. The method according to claim 1, wherein said subject is a subject that suffered from a primary cancer and in which the primary cancer was surgically resected.

5. The method according to claim 1, wherein said sample comprising peptides from a subject is a bodily fluid sample from said subject.

6. The method according to claim 1, wherein said sample comprising peptides is a sample comprising collagen natural occurring peptides (NOPs).

7. The method according to claim 2, wherein said reference peptide level is measured in a sample comprising peptides from a reference subject not suffering from, or a reference subject not having suffered from, cancer.

8. The method according to claim 7, wherein said subject, or said sample, is typed as having a secondary liver cancer when said peptide level is increased as compared to said reference peptide level.

9. The method according to claim 1, further comprising the steps of
measuring in a sample comprising proteins from said subject a carcinoembryonic antigen (CEA) protein level;
typing said subject for the presence or absence of a secondary liver cancer on the basis of the measured peptide level and the measured CEA protein level.

10. The method according to claim 9, wherein said proteins from said subject are proteins from a blood sample of said subject.

11. The method according to claim 9, further comprising the steps of
comparing said measured protein level to a reference CEA protein level; and
typing said subject for the presence or absence of a secondary liver cancer on the basis of the (i) comparison of the measured peptide level and the reference peptide level and (ii) comparison of the measured CEA protein level and the reference CEA protein level.

12. The method according to claim 11, wherein said reference CEA protein level is measured in a sample comprising proteins from a reference subject not suffering from, or a reference subject not having suffered from, cancer.

13. The method according to claim 12, wherein said subject, or said sample, is typed as having a secondary liver cancer when (i) said peptide level is increased as compared to said reference peptide level and (ii) said CEA protein level is increased as compared to said reference CEA protein level.

14. (canceled)

15. The method according to claim 1 wherein said secondary liver cancer is colorectal liver metastases (CRLM).

16. A peptide comprising the amino acid sequence of SEQ ID NO:4 or a peptide comprising an amino acid sequence that has at least 90% sequence identity to the amino acid sequence of SEQ ID NO:4, or a peptide comprising the amino acid sequence of SEQ ID NO:1 or a peptide comprising an amino acid sequence that has at least 90% sequence identity to the amino acid sequence of SEQ ID NO:1.

17. A peptide according to claim 16, wherein said peptide comprises the amino acid sequence of SEQ ID NO:4 or SEQ ID NO:1.

18. A standard-of-care therapeutic agent against a secondary liver cancer for use in treating a subject typed as having a secondary liver cancer according to the method of claim 1.

19. A method for treating a subject suffering from a secondary liver cancer, comprising the step of
performing a method according to claim 1;
administering a therapeutically effective amount of a standard-of-care therapeutic agent against secondary liver cancer when said subject is typed as having a secondary liver cancer.

20. A method for measuring a peptide level, comprising the step of:
optionally, providing a sample comprising peptides from a subject;
measuring in a sample comprising peptides from a subject a peptide level for (i) a peptide comprising the amino acid sequence of SEQ ID NO:4 or a peptide comprising an amino acid sequence that has at least 90% sequence identity to the amino acid sequence of SEQ ID NO:4, and/or (ii) a peptide comprising the amino acid sequence of SEQ ID NO:1 or a peptide comprising an amino acid sequence that has at least 90% sequence identity to the amino acid sequence of SEQ ID NO:1.

21. The method according to claim 1, wherein the subject is a subject suffering from, or a subject having suffered from, a primary colorectal cancer.

22. The method according to claim 1, wherein said sample comprising peptides from a subject is a urine sample from said subject.

23. A method for treating a subject suffering from a secondary liver cancer, comprising the step of
performing a method according to claim 1;
administering a therapeutically effective amount of a standard-of-care therapeutic agent against secondary liver cancer when said subject is typed as having colorectal liver metastases.


SEQUENCE LISTING (6 sequences)

SEQ ID NO: 1 - protein, 35 aa, Artificial Sequence
  Hydroxylated GND peptide
  MOD_RES (15)..(15): hydroxyproline
  MOD_RES (17)..(18): hydroxyproline
  MOD_RES (24)..(24): hydroxyproline
  MOD_RES (27)..(27): hydroxylproline
  MOD_RES (30)..(30): Hyl
GNDGARGSDGQPGPPGPPGTAGFPGSPGAKGEVGP

SEQ ID NO: 2 - protein, 23 aa, Artificial Sequence
  Hydroxylated GPP peptide
  MOD_RES (8)..(8): Hyl
  MOD_RES (9)..(9): hydroxyproline
  MOD_RES (15)..(15): hydroxyproline
  MOD_RES (21)..(21): hydroxyproline
GPPGEAGKPGEQGVPGDLGAPGP

SEQ ID NO: 3 - protein, 24 aa, Artificial Sequence
  Hydroxylated AGP peptide
  MOD_RES (4)..(4): hydroxyproline
  MOD_RES (10)..(10): hydroxyproline
  MOD_RES (16)..(16): hydroxyproline
  MOD_RES (22)..(22): hydroxyproline
AGPPGEAGKPGEQGVPGDLGAPGP

SEQ ID NO: 4 - protein, 35 aa, Artificial Sequence
  Hydroxylated GER peptide
  MISC_FEATURE (6)..(6): hydroxyproline
  MISC_FEATURE (9)..(9): hydroxyproline
  MISC_FEATURE (15)..(15): hydroxyproline
  MISC_FEATURE (21)..(21): hydroxyproline
  MISC_FEATURE (24)..(24): hydroxyproline
  MISC_FEATURE (33)..(33): hydroxyproline
  MISC_FEATURE (35)..(35): hydroxyproline
GERGSPGGPGAAGFPGARGLPGPPGSNGNPGPPGP

SEQ ID NO: 5 - protein, 24 aa, Artificial Sequence
  (SIL) peptide
  MISC_FEATURE (4)..(4): hydroxyproline
  MISC_FEATURE (9)..(9): labelled with 13C615N2
  MISC_FEATURE (10)..(10): hydroxyproline
  MISC_FEATURE (16)..(16): hydroxyproline
  MISC_FEATURE (22)..(22): hydroxyproline
AGPPGEAGKPGEQGVPGDLGAPGP

SEQ ID NO: 6 - protein, 35 aa, Artificial Sequence; identical to SEQ ID NO: 4
  NOP GER
  MOD_RES (6)..(6): 4Hyp
  MOD_RES (9)..(9): 4Hyp
  MOD_RES (15)..(15): 4Hyp
  MOD_RES (21)..(21): 4Hyp
  MOD_RES (24)..(24): 4Hyp
  MOD_RES (33)..(33): 4Hyp
  MOD_RES (35)..(35): 4Hyp